(19) United States
(12) Patent Application Publication
(10) Pub. No.: US 2008/0194560 A1
(43) Pub. Date: Aug. 14, 2008

(54) DISINTEGRATION PROMOTERS IN SOLID DOSE WET GRANULATION FORMULATIONS

(76) Inventors: Zhi Yun Wang, East Hanover, NJ (US); David Harris, New Providence, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(21) Appl. No.: 11/960,320

(22) Filed: Dec. 19, 2007

Related U.S. Application Data
(60) Provisional application No. 60/871,605, filed on Dec. 22, 2006.

(51) Int. Cl. A61K 31/5355; A61K 31/443; A61K 31/444; A61P 9/00

(52) U.S. Cl. 514/235.2; 514/333; 514/337

(57) ABSTRACT

Disclosed is a rapidly disintegrating solid dosage form comprising an active pharmaceutical ingredient, a disintegrant and a disintegration promoter, which formulation is obtainable by a wet granulation process. In one exemplified embodiment, the active pharmaceutical agent is a thrombin receptor antagonist, the disintegrant is sodium croscarmellose, and the disintegration promoter is calcium silicate. In some embodiments, the thrombin receptor antagonist is represented by the formula:

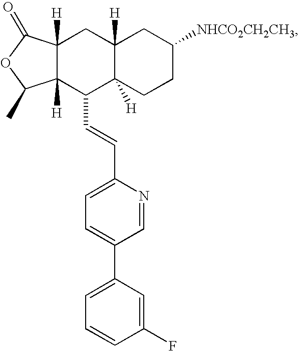

or a pharmaceutically acceptable salt thereof.

Also disclosed are methods of treating patients at risk for acute coronary syndrome by administering such a rapidly disintegrating solid dosage form.

### CROSS-REFERENCE TO RELATED APPLICATIONS

This application claims the benefit of provisional application No. 60/871,605, filed Dec. 22, 2006, which application is herein incorporated in its entirety.

### FIELD OF THE INVENTION

The present invention relates to the use of a disintegration promoter in conjunction with a disintegrant in rapidly disintegrating orally administered pharmaceutical formulations prepared by wet granulation.

### BACKGROUND OF THE INVENTION

Rapid disintegration of an orally administered pharmaceutical dosage form can be critical where there is a need to quickly raise the blood concentration level of an active pharmaceutical ingredient in a patient. Such a need may arise in a patient who is suffering from an acute condition that is treatable by the active pharmaceutical ingredient, and who may be at near-term risk for more serious events if left untreated. Acute Coronary Syndrome (“ACS”) is such a condition.

ACS is an umbrella term used to cover any group of clinical symptoms compatible with acute myocardial ischemia, including unstable angina, and non-ST segment elevation myocardial infarction (MI) and ST segment elevation MI. Acute myocardial ischemia is associated with chest pain due to insufficient blood supply to the heart muscle that results from coronary artery disease (also called coronary heart disease). These life-threatening disorders are a major cause of emergency medical care and hospitalization in the United States. Coronary heart disease is the leading cause of death in the United States. Unstable angina and non-ST-segment elevation myocardial infarction are very common manifestations of this disease. Schering-Plough Corp. is currently developing a thrombin receptor antagonist for the indication of ACS. A rapidly disintegrating orally administered loading dose of the thrombin receptor antagonist is within this development program.

Thrombin receptor antagonists (“TRAs”) have been suggested in the literature as being potentially useful in treating a variety of cardiovascular diseases or conditions including, for example, thrombosis, vascular restenosis, deep venous thrombosis, lung embolism, cerebral infarction, heart disease, disseminated intravascular coagulation syndrome, hypertension (Suzuki, Shuichi, PCT Int. Appls. WO 0288092, WO 0285850 and WO 0285855), arrhythmia, inflammation, angina, stroke, atherosclerosis, ischemic conditions (Zhang, Han-cheng, PCT Int. Appl. WO 0100659, WO 0100657 and WO 0100656).

Thrombin receptor antagonists are disclosed in U.S. Pat. Nos. 6,063,847; 6,326,380; and 6,645,987, and in U.S. publication nos. 03/0203927; 04/0216437A1; 04/0152736; and U.S. Pat. No. 7,304,078. The use of a small subset of thrombin receptor antagonists to treat a variety of conditions and diseases is disclosed in U.S. publication no. 04/0192753. A bisulfate salt of a particular thrombin receptor antagonist is disclosed in U.S. Pat. No. 7,235,567. All of these patents and patent publications mentioned herein are incorporated by reference in their entirety.

A variety of formulation technologies are known to provide solid dosage forms that rapidly disintegrate to release the active ingredient in the oral cavity. Among these formulation technologies is that of wet granulation. Wet granulation formulations of thrombin receptor antagonists are disclosed in U.S. application Ser. Nos. 11/771,520, 11/771,571 and 11/860,165.

Wet granulation technologies are often used to process powders prior to compaction in a tableting step. The application of wet granulation technology for the preparation of a rapidly disintegrating solid oral dosage form requires the selection of appropriate excipients and fine-tuning of process conditions to result in a tablet that exhibits sufficient disintegration times to qualify as a rapidly disintegrating formulation, but with sufficient friability to withstand the mechanical stresses imparted by packaging and normal handling without losing structural integrity. Furthermore, the formulation must not result in a solid dosage form that is too large for convenient oral administration. The selection of the excipient list and the amount of each in a wet granulation formulation can be crucial to achievement of a dosage form that exhibits the required disintegration time, structural integrity and tablet size. Thus, there exists the need for improvements to wet granulation formulations to achieve these goals.

### SUMMARY OF THE INVENTION

In one embodiment, the invention is directed to a rapidly disintegrating solid dosage form comprising an active pharmaceutical ingredient, a disintegrant and a disintegration promoter, wherein said dosage form is the product of a wet granulation process.

In some embodiments, the disintegration promoter is calcium silicate.

In some embodiments, the disintegrant is sodium croscarmellose.

In some embodiments, the disintegrant comprises about 5 wt % to about 10 wt %. of the solid dosage form.

In some embodiments, the disintegration promoter comprises about 5 wt % to about 50 wt % of said solid dosage form.

In some embodiments, the ratio of the weights of disintegrant to disintegration promoter is between about 0.2 and about 0.5.

In some embodiments, the active pharmaceutical ingredient is a thrombin receptor antagonist. In some embodiments, an average platelet inhibition of at least about 80% is achieved within 30 minutes of administration. In some embodiments, the thrombin receptor antagonist is represented by the formula:

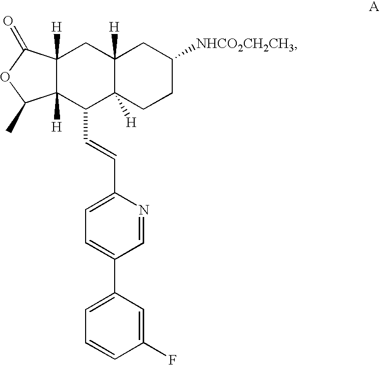

or a pharmaceutically acceptable salt thereof. In some embodiments, the pharmaceutically acceptable salt is the bisulfate salt.

In some embodiments, the thrombin receptor antagonist is represented by the formula:

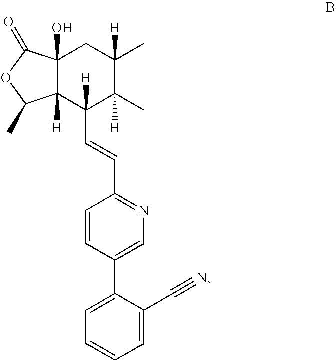

or a pharmaceutically acceptable salt thereof.

In some embodiments, the thrombin receptor antagonist is represented by the formula:

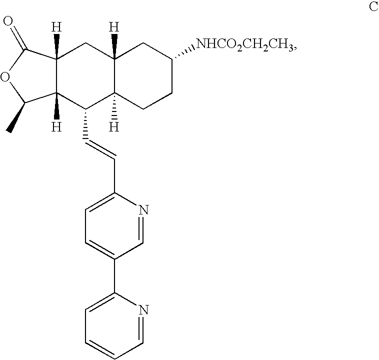

or a pharmaceutically acceptable salt thereof.

In some embodiments, the thrombin receptor antagonist is represented by the formula:

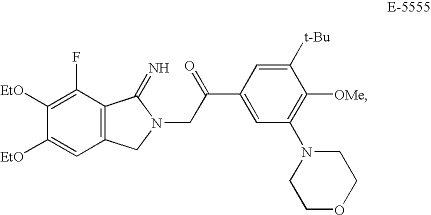

or a pharmaceutically acceptable salt thereof.

In some embodiments, the rapidly disintegrating solid dosage form further comprises a binder. In some embodiments, the binder is povidone.

In some embodiments, the rapidly disintegrating solid dosage form further comprises a first filler. In some embodiments, the first filler is microcrystalline cellulose.

In some embodiments, the rapidly disintegrating solid dosage further comprises a second filler. In some embodiments, the second filler is mannitol.

In some embodiments, the rapidly disintegrating solid dosage form further comprises a lubricant. In some embodiments, the lubricant is magnesium stearate.

In some embodiments, the dosage form disintegrates within about 30 seconds of placement in the oral cavity.

In some embodiments, the dosage form disintegrates within about 15 seconds of placement in the oral cavity.

In some embodiments, the dosage form disintegrates within about 10 seconds of placement in the oral cavity.

In one embodiment, the invention is directed to a method of treating a patient at risk of acute coronary syndrome comprising administering the rapidly disintegrating solid dosage form comprising a thrombin receptor antagonist. In one embodiment, the thrombin receptor antagonist is represented by the formula:

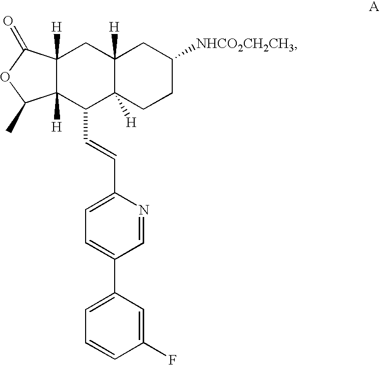

or a pharmaceutically acceptable salt thereof. In some embodiments, the pharmaceutically acceptable salt is the bisulfate salt.

### DETAILED DESCRIPTION OF THE INVENTION

As part of its TRA development program, Schering-Plough Corp. has investigated improvements to its wet granulation tablet formulations. These formulations include sodium croscarmellose as a disintegrant. The question arose as to whether a properly selected excipient could act as a disintegration promoter, and thus boost the disintegration action imparted to the tablet by the disintegrant. In particular, the use of calcium silicate as a disintegration promoter was investigated.

As used herein, the term “granulation” refers to the process of agglomerating powder particles into larger granules that contain the active pharmaceutical ingredient. The term “wet granulation” refers to any process comprising the steps of addition of a liquid to powdered starting materials, agitation, and drying to yield a solid dosage form. The resulting granulated drug product may be further processed into various final dosage forms, e.g., capsules, tablets, wafers, gels, lozenges, etc.

In typical rapidly disintegrating formulations, disintegration times after oral administration are less than 30 seconds, preferably less than 15 seconds, more preferably, less than 10 seconds. Dissolution times are typically measured in an in vitro setting using pharmaceutical compendial apparatus such as the USP Dissolution Apparatus 1 (basket) or Apparatus 2 (paddle). Alternate dissolution test methodologies may also be employed, e.g., flow-through dissolution cells, based upon the physical nature of the embodiment.

The raw material in a wet granulation process is typically the active pharmaceutical ingredient in a powder form. The powder can be generated by grinding, and the particle size distribution that results from the grinding step may influence the properties of the formulation. The active pharmaceutical ingredient may be mixed with other excipients, such as binders, disintegrants, fillers, or lubricants, into a powder blend.

The powder blend is then introduced to a granulation fluid, which is typically applied by spraying for the most uniform distribution of liquid. The relative quantity of granulation fluid applied will influence the size and mechanical properties of the granules that form. An excess of granulation fluid can result in formation of a slurry that may present problems in later processing, e.g., wet sieving. The granulation fluid may be aqueous or non-aqueous, depending on such physicochemical properties of the active pharmaceutical ingredient as solubility. Various excipients, e.g., binders, disintegrants, fillers, or lubricants, may be mixed into the granulation fluid prior to application to the powder blend. The granulation fluid is typically applied to the powder blend in a closed vessel, usually with agitation. To ensure complete mixing, high-shear agitation may be applied.

After the granulation fluid has been applied to the powder mix, and sufficient agitation has been applied to agglomerate the powder particles into granules, the granules are dried and milled. After milling, additional excipients may be added to form the final blend. Such additional excipients may include binders, disintegrants, fillers, and lubricants. The final blend is then compressed into the desired solid dosage form, e.g., tablets.

The bisulfate salt of Compound A, a high activity thrombin receptor antagonist, was used as the active pharmaceutical ingredient in a rapidly disintegrating formulation prepared using a wet granulation technique.

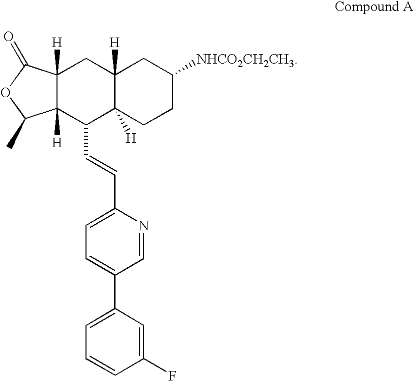

Table 1 displays the components used in this wet granulation.

TABLE 1
Components Used in Exemplified Wet Granulation

| | Component | Function | Wt. (g) | |
|---|---|---|---|---|
| Powder | Compound A bisulfate | API | 20.0 | |
| Blend | Sodium Croscamellose | Disintegrant | 2.0 | |
| | Microcrystalline Cellulose | 1^{st} Filler | 27.0 | |
| | (Avicel PH 102) | | | |
| Granulation | Povidone K30 | Binder | 1.0 | |
| Fluid | Deionized water | — | 325 | mL^{a} |
| Additional | Calcium Silicate (FM1000) | Disintegration | 27.0 | |
| Excipients | | Promoter | | |
| | Sodium Croscamellose | Disintegrant | 7.0 | |
| | Microcrystalline Cellulose | 1^{st} Filler | 10.0 | |
| | (Avicel PH 102) | | | |
| | Mannitol (direct | 2^{nd} Filler | 29.0 | |
| | compression) | | | |
| | Magnesium Stearate | Lubricant | 2.0 | |
| | Total Core Weight | | 125.0 | |

^{a}Evaporated.

A powder mix was prepared comprising Compound A bisulfate, povidone K30 (a binder), microcrystalline cellulose (Avicel PH 102, a filler) and sodium croscarmellose (a disintegrating agent). A granulation fluid was prepared by dissolving povidone in deionized water. The granulation fluid was sprayed into the powder blend in a high shear granulator. The resultant granulation was dried, milled and blended with additional excipients including calcium silicate, sodium croscarmellose, microcrystalline cellulose, mannitol and magnesium stearate using a conventional tumble blender. The final blend was compressed into tablets using a rotary tablet press. Friability and disintegration tests were performed to evaluate tablet performance. Here, calcium silicate was used as a disintegration promoter, sodium croscarmellose as a disintegrant, microcrystalline cellulose as a second filler, magnesium stearate as a lubricant, and mannitol as used as a filler and to promote good mouth-feel. The resultant formulation is displayed in Table 2.

TABLE 2
Exemplified Wet Granulation Final Formulation

| Compositions | Wt. (g) | Wt. Percent |
|---|---|---|
| Compound A Bisulfate | 20.0 | 16.0 |
| Sodium Croscamellose | 9.0 | 7.2 |
| Microcrystalline Cellulose (Avicel | 37.0 | 29.6 |
| PH 102) | | |
| Povidone K30 | 1.0 | 0.8 |
| Calcium Silicate (FM1000) | 27.0 | 21.6 |
| Mannitol (direct compression) | 29.0 | 23.2 |
| Magnesium Stearate | 2.0 | 1.6 |
| Total Core Weight | 125.0 | 100.0 |

As used herein, the term “disintegration promoter” means an excipient whose presence in the formulation along with that of a disintegrant, boosts the rate of disintegration of the dosage form relative to a formulation containing the same amount of disintegrant, but none of the excipient. In wet granulation formulations using sodium croscarmellose as a disintegrant, calcium silicate is an example of a disintegration promoter. In the present invention, the disintegration promoter preferably comprises between about 5 wt % and about 50 wt % of the solid dosage form, and the ratio of the weights of disintegrant to disintegration promoter is between about 0.2 and about 0.5.

In the above formulation, calcium silicate and sodium croscarmellose comprised about 29% of the final blend. The calcium silicate to sodium croscarmellose ratio was 27:7. Such high contents of calcium silicate and sodium croscarmellose did not negatively impact processibility of the formulation. As reflected in Table 3, at a tablet hardness of 3-4 KP, disintegration time was measured at 8-10 seconds in a USP Disintegration Tester using 900 mL deionized water at 37° C. The fast tablet disintegration time was not compromised by increases of tablet hardness and was not associated with extremely soft tablets or tablet defects. Friability of tablets was low, i.e., ≦0.15%.

TABLE 3
Disintegration Times and Friabilities

| | |
|---|---|
| Low Hardness | High Hardness |
| (3-4 KP) | (4.5-6 KP) |

| Disintegration | Friability | Disintegration | Friability |
|---|---|---|---|
| 8-10 seconds | 0.15% | 9-11 seconds | 0% |
| (N = 3) | | (N = 3) | |

Tablets containing no disintegration promoter (e.g., calcium silicate) exhibited much slower disintegration times. For example, a tablet formulation containing 29% sodium croscarmellose and 0% calcium silicate had an average disintegration time of 5 minutes. Therefore, it was concluded that appropriate use of a disintegrant and disintegration promoter achieved fast disintegrating tablets for Compound A bisulfate.

Each of the excipients referenced above has a particular function in Xs either the wet granulation process or in the dosage form. Binders are typically polymers used as granulating agents to increase granule strength sufficiently to withstand the drying process.

As used herein, the term “polymer” shall be understood to include gelatins, modified starches, materials derived from animal or vegetable proteins, dextrins and soy, wheat and psyllium seed proteins; gums such as acacia, guar, agar, and xanthan; polysaccharides; alginates; carboxymethylcelluloses; carrageenans; dextrans; pectins; synthetic polymers such as polyvinylpyrrolidone; and polypeptide/protein or polysaccharide complexes such as gelatin-acacia complexes.

A range of modified starches are commercially available and useful in the present invention and include:

Pregelatinized starches, produced by drum drying or extrusion;

Low-viscosity starches, produced by controlled hydrolysis of glycosidic bonds;

Dextrins, produced by roasting dry starch in the presence of a small amount of acid;

Acid modified starches, produced by suspension in dilute acid until the required viscosity is reached;

Oxidized starches, in which oxidizing agents cause the introduction of carbonyl or carboxyl groups, wherein depolymerization occurs, leading to decreased retrogradation and gelling capacities;

Enzymatically modified starch, produced by controlled enzyme degradation to attain required physicochemical properties;

Crosslinked starches, generated by reacting bi- or polyfunctional reagents (e.g., phosphorus oxychloride, sodium trimetaphosphate and epichlorohydrin) with hydroxyl groups to form crosslinks; and,

Stabilized starches, produced by reacting a starch with etherifying or esterifying reagents in the presence of an alkaline catalyst to give a wide range of products.

Commonly used binders include starch, pre-gelatinized starch, acacia, polyvinylpyrrolidone (“PVP”), and hydroxypropyl methylcellulose (“HPMC”).

Disintegrants are used to promote swelling and disintegration of the tablet after exposure to fluids in the oral cavity. Commonly used disintegrants include starch, microcrystalline cellulose, insoluble ion exchange resins, sodium starch glycolate, croscarmellose sodium, alginic acid, sodium alginate, crospovidone and gums, including agar, guar and xanthan. In the present invention, the disintegrant preferably comprises between about 5 wt % and about 10 wt % of the solid dosage form.

Lubricants are used to promote flowability of powders, and to reduce friction between the tablet punch faces and the tablet punches and between the tablet surface and the die wall. Among the most commonly used lubricants are magnesium stearate, calcium stearate, sodium stearyl fumarate, polyethylene glycol, sodium lauryl sulphate magnesium lauryl sulphate, and sodium benzoate.

Fillers provide bulk and can bind to the active pharmaceutical ingredient, thus reducing the potential for segregation and promoting content uniformity. Commonly used fillers include microcrystalline cellulose, starch, dibasic calcium phosphate dihydrate, lactose, sorbitol, and mannitol.

The present invention encompasses the use of disintegration promoters in wet granulation formulations comprising any compatible active pharmaceutical ingredient, including but not limited to thrombin receptor antagonists. However, in view of the utility of TRAs in treating ACS, and the criticality of rapid dosing of patients at risk for ACS, the inventor conceives her invention as particularly encompassing the use of any and all thrombin receptor antagonists.

A variety of compounds have been demonstrated as displaying activity as thrombin receptor antagonists, many being himbacine analogs. As disclosed in U.S. publication no. 04/0152736, a subset of particularly preferred compounds of Formula I is as follows:

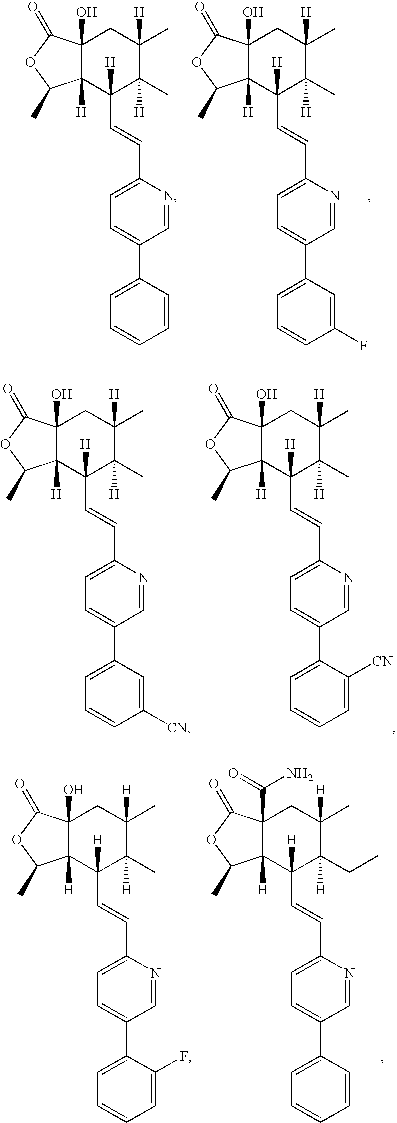

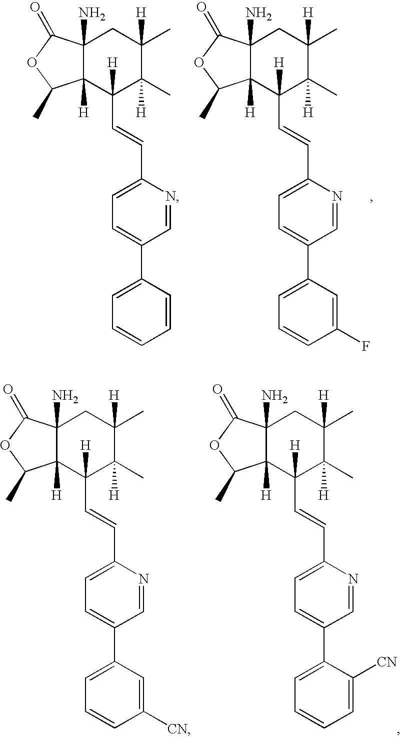

and pharmaceutically acceptable salts thereof.

U.S. Pat. No. 7,304,078 discloses a subset of thrombin receptor antagonists of Formula II which are both particularly active and selective. These compounds are as follows:

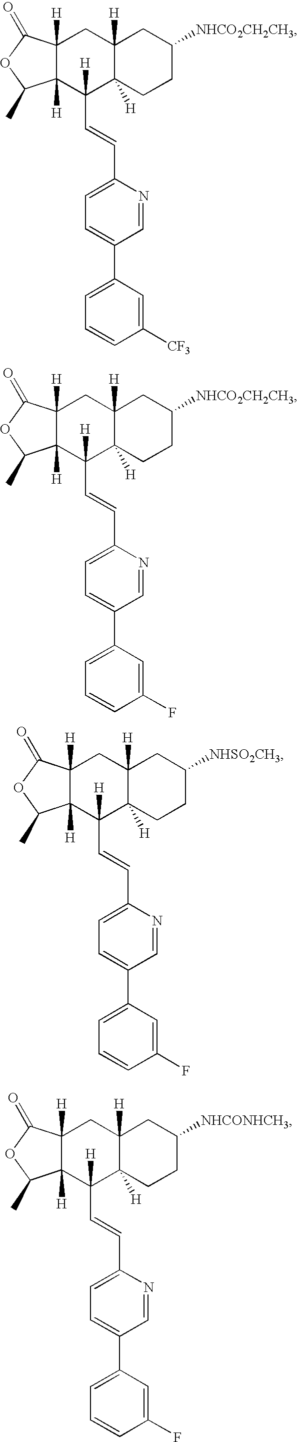

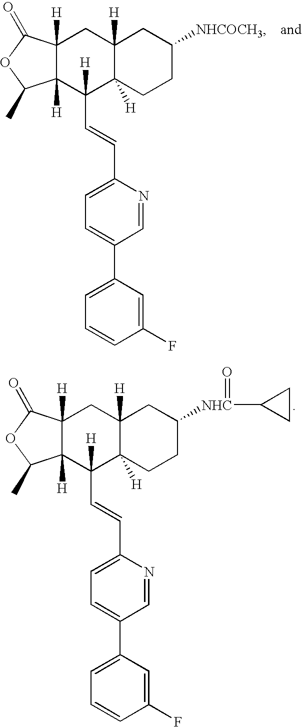

and the pharmaceutically acceptable isomers, salts, solvates and polymorphs thereof.

The following compounds are particularly favored based on their pharmacokinetics and pharmacodynamic characteristics:

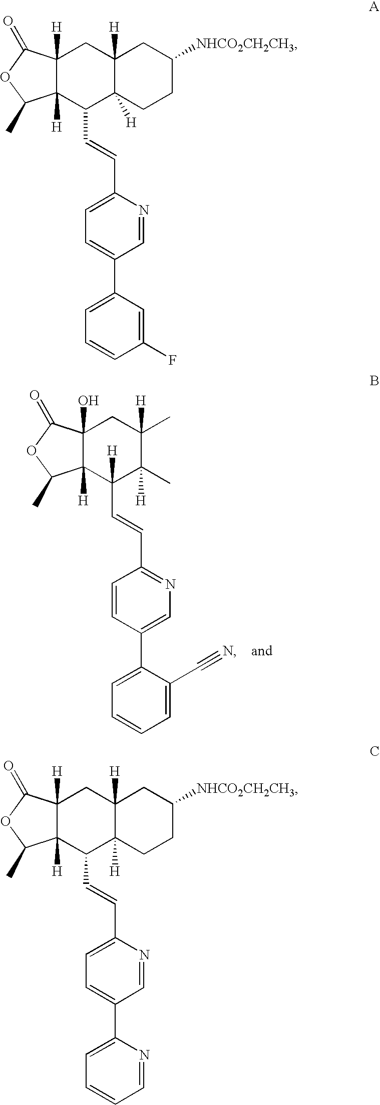

or a pharmaceutically acceptable isomer, salt, solvate or co-crystal form thereof. Compounds A and C are disclosed in U.S. Pat. No. 7,304,078, and the bisulfate salt of Compound A is disclosed in U.S. Pat. No. 7,235,567. The bisulfate salt of Compound A is currently in development as a thrombin receptor antagonist by Schering-Plough Corp. Compound B is disclosed in U.S. Pat. No. 6,645,987.

Other compounds for use in the formulations of the present invention are disclosed in any of U.S. Pat. Nos. 6,063,847, 6,326,380, U.S. patent Publications U.S. Ser. No. 03/0,203,927, U.S. Ser. No. 03/0,216,437, U.S. Ser. No. 04/0,192,753, and U.S. Ser. No. 04/0,176,418, the compound-related disclosures of which are all incorporated by reference in their entirety. Formulations that include other agents that display activity as thrombin receptor antagonists are also within the scope of the present invention, including E5555 currently in development by Eisai, the structure of which is as follows:

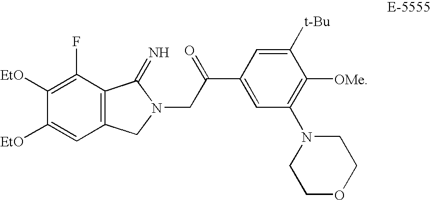

A series of indazole peptidomimetics is reported as displaying activity as thrombin receptor antagonists in U.S. Pat. No. 7,049,297, which is incorporated herein by reference in its entirety.

In one embodiment of the present invention, the formulation is an oral solid dosage form that can be swallowed without water, because it disintegrates rapidly on the tongue, in some embodiments, in less than about 30 seconds, preferably, in less than about 15 seconds, more preferably, in less than about 10 seconds. Such a rapid disintegration phenomenon may provide for enhanced dissolution of the active ingredient, and subsequent realization of an optimal, i.e., rapid, pharmacokinetic profile of such an ingredient. Preferably, essentially all of the thrombin receptor antagonist dissolves within about 15 minutes.

One of the ultimate purposes in providing a rapidly disintegrating solid dosage form is to provide a blood concentration profile of the thrombin receptor antagonist sufficient to result in a rapid onset of blood platelet inhibition in the patient at risk for ACS. The TRA embodiments of the present invention are believed to result in an average platelet inhibition of at least about 80% within 30 minutes of administration. Platelet inhibition is discussed in U.S. Pat. No. 7,304,078 at cols. 52-54, which discussion is incorporated herein.

The present invention further encompasses methods of treatment of a patient at risk of Acute Coronary Syndrome by administering an effective amount of a rapidly disintegrating formulation of a thrombin receptor antagonist as described above. As used herein, the term “effective amount” will be understood to describe an amount of a thrombin receptor antagonist effective to prevent further damage to the cardiovascular system after an acute cardiac event. The rapidly disintegrating dosage forms described herein are contemplated for use in administration of a loading dose of a thrombin receptor antagonist. The formulations of the present invention preferably contain a thrombin receptor antagonist described above in an amount of about 10 mg to about 50 mg. Doses of 10, 20 and 40 mg are candidates for the thrombin receptor loading dose. A 40 mg loading dose is planned for administration in phase III clinical trials.

While the present invention has been described in conjunction with the specific embodiments set forth above, many alternatives, modifications and variations thereof will be apparent to those of ordinary skill in the art. All such alternatives, modifications, and variations are intended to fall within the spirit and scope of the present invention.

What is claimed is:

1. A rapidly disintegrating solid dosage form comprising an active pharmaceutical ingredient, a disintegrant and a disintegration promoter, wherein said dosage form is the product of a wet granulation process.

2. The rapidly disintegrating solid dosage form of claim 1, wherein the disintegration promoter is calcium silicate.

3. The rapidly disintegrating solid dosage form of claim 1, wherein the disintegrant is sodium croscarmellose.

4. The rapidly disintegrating solid dosage form according to claim 1, wherein said disintegrant comprises about 5 wt % to about 10 wt %. of said solid dosage form.

5. The rapidly disintegrating solid dosage form according to claim 1, wherein said disintegration promoter comprises about 5 wt % to about 50 wt % of said solid dosage form.

6. The rapidly disintegrating solid dosage form according to claim 1, wherein the ratio of the weights of disintegrant to disintegration promoter is between about 0.2 and about 0.5.

7. The rapidly disintegrating solid dosage form according to claim 1, wherein the active pharmaceutical ingredient is a thrombin receptor antagonist.

8. The rapidly disintegrating solid dosage form according to claim 7, wherein the thrombin receptor antagonist is represented by the formula: 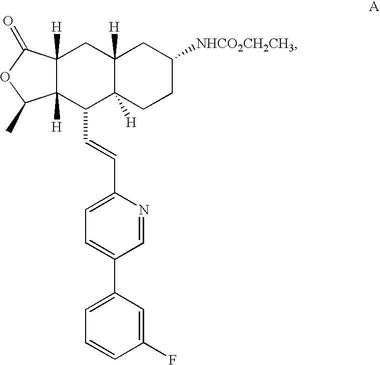
or a pharmaceutically acceptable salt thereof.

9. The rapidly disintegrating solid dosage form according to claim 8, wherein the pharmaceutically acceptable salt is the bisulfate salt.

10. The rapidly disintegrating solid dosage form according to claim 7, wherein the thrombin receptor antagonist is represented by the formula: 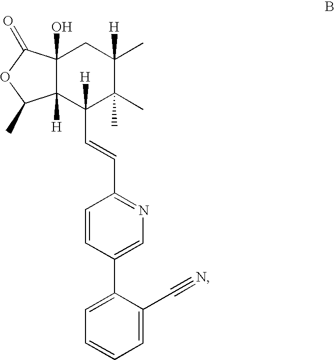
or a pharmaceutically acceptable salt thereof.

11. The rapidly disintegrating solid dosage form according to claim 7, wherein the thrombin receptor antagonist is represented by the formula: 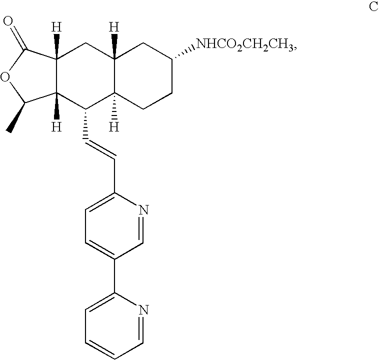
or a pharmaceutically acceptable salt thereof.

12. The rapidly disintegrating solid dosage form according to claim 7, wherein the thrombin receptor antagonist is represented by the formula: 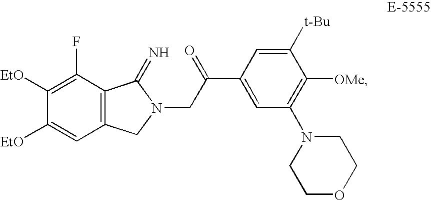
or a pharmaceutically acceptable salt thereof.

13. The rapidly disintegrating solid dosage form according to claim 1, further comprising a binder.

14. The rapidly disintegrating solid dosage form according to claim 13, wherein said binder is povidone.

15. The rapidly disintegrating solid dosage form according to claim 1, further comprising a first filler.

16. The rapidly disintegrating solid dosage form according to claim 15, wherein said first filler is microcrystalline cellulose.

17. The rapidly disintegrating solid dosage form according to claim 16, further comprising a second filler.

18. The rapidly disintegrating solid dosage form according to claim 17, wherein said second filler is mannitol.

19. The rapidly disintegrating solid dosage form according to claim 1, further comprising a lubricant.

20. The rapidly disintegrating solid dosage form according to claim 19, wherein said lubricant is magnesium stearate.

21. A method of treating a patient at risk of acute coronary syndrome comprising administering the rapidly disintegrating solid dosage form of claim 7.

22. A method of treating a patient at risk of acute coronary syndrome comprising administering the rapidly disintegrating solid dosage form of claim 8.

23. A method of treating a patient at risk of acute coronary syndrome comprising administering the rapidly disintegrating solid dosage form of claim 9.

24. A method of treating a patient at risk of acute coronary syndrome comprising administering the rapidly disintegrating solid dosage form of claim 10.

25. A method of treating a patient at risk of acute coronary syndrome comprising administering the rapidly disintegrating solid dosage form of claim 12.
